(19) United States
(12) Patent Application Publication
(10) Pub. No.: US 2020/0289687 A1
(43) Pub. Date: Sep. 17, 2020

(54) Ultraviolet Light Sanitation Device for Retail Checkout Counter Conveyor Belts

(71) Applicants: Kelsey Marie Riley, West Haven, UT (US); Daren Karl Brooks, West Haven, UT (US)

(72) Inventors: Kelsey Marie Riley, West Haven, UT (US); Daren Karl Brooks, West Haven, UT (US)

(21) Appl. No.: 16/819,040

(22) Filed: Mar. 13, 2020

Related U.S. Application Data
(60) Provisional application No. 62/817,897, filed on Mar. 13, 2019.

(51) Int. Cl. A61L 2/10; A47F 9/04

(52) U.S. Cl. CPC A61L 2/10; A47F 2009/041; A47F 9/04

(57) ABSTRACT

Disclosed is an ultraviolet light sanitation device for retail checkout counter conveyor belts with a UV-C light source, a housing, and a mount. The UV-C light source is internally positioned at an apex end of the housing opposite a treatment opening of the housing, where a conveyor belt of a retail checkout counter is exposed to the UV-C light source through the treatment opening in order to sanitize the conveyor belt through germicidal irradiation by the UV-C light source. The mount connects the housing to a retail checkout counter such that the treatment opening is positioned adjacent to the conveyor belt, either adjacent to an underside of the conveyor belt within a conveyor cabinet of the retail checkout counter, or external to and atop the conveyor cabinet.

The current application claims a priority to the U.S. Provisional Patent application Ser. No. 62/817,897 filed on Mar. 13, 2019.

### FIELD OF THE INVENTION

The present invention relates generally to sanitation. More particularly, the present invention relates to ultraviolet light sanitation of conveyor belts.

### BACKGROUND OF THE INVENTION

Until now, there has been no solution for the need of cleansing and antiseptic for belts of all types, more particularly the belts used to move food at grocery stores, warehouses, food service establishments, restaurants, and the like. This device encompasses a disinfecting and cleansing apparatus that can be either installed at the manufacturing level or retrofitted to existing equipment to remedy this current problem and meet this current need.

It has been well documented that UV light, when shined with proper duration and intensity has excellent antiseptic properties. In addition to the need for antiseptic treatment on these food carrying belts and conveyors, there is also a need to clean actual debris as well as larger particles from the belt as well.

Additional advantages of the invention will be set forth in part in the description which follows, and in part will be obvious from the description, or may be learned by practice of the invention. Additional advantages of the invention may be realized and attained by means of the instrumentalities and combinations particularly pointed out in the detailed description of the invention section. Further benefits and advantages of the embodiments of the invention will become apparent from consideration of the following detailed description given with reference to the accompanying drawings, which specify and show preferred embodiments of the present invention.

### BRIEF DESCRIPTION OF THE DRAWINGS

FIG. 1 is a perspective view of one embodiment of the present invention configured to be installed underneath a conveyor belt.

FIG. 2 is a diagram of various electrical components of the present invention.

FIG. 3 is a lowered perspective view of one embodiment of the present invention configured to be installed atop a conveyor belt.

FIG. 4 is a top view of the housing in some embodiments wherein the housing is adjustable in length.

FIG. 5 is an exploded cross-sectional view along line A-A of FIG. 4.

### DETAIL DESCRIPTIONS OF THE INVENTION

All illustrations of the drawings are for the purpose of describing selected versions of the present invention and are not intended to limit the scope of the present invention. The present invention is to be described in detail and is provided in a manner that establishes a thorough understanding of the present invention. There may be aspects of the present invention that may be practiced or utilized without the implementation of some features as they are described. It should be understood that some details have not been described in detail in order to not unnecessarily obscure focus of the invention. References herein to “the preferred embodiment”, “one embodiment”, “some embodiments”, or “alternative embodiments” should be considered to be illustrating aspects of the present invention that may potentially vary in some instances, and should not be considered to be limiting to the scope of the present invention as a whole.

The present invention is an ultraviolet (UV) sanitation device for retail checkout counter conveyor belts. The present invention may be installed onto an existing retail checkout counter, or may be integrated into a retain checkout counter during its manufacture. The present invention utilizes a high intensity short-wavelength UV-C light source to sanitize the conveyor belt as it moves relative to the present invention during normal operation through ultraviolet germicidal irradiation, which functions to inactivate any microorganisms such as bacteria and viruses which may be present on the surface of the conveyor belt. Though the present invention is intended for use with conveyor belts of retail checkout counters, such as those found in supermarkets, the present invention may be used with any applicable variety of conveyor belt, and may be more generally used in any relevant application where a surface or other region is desired to be sanitized through ultraviolet germicidal irradiation from a short wavelength UV-C light source 1.

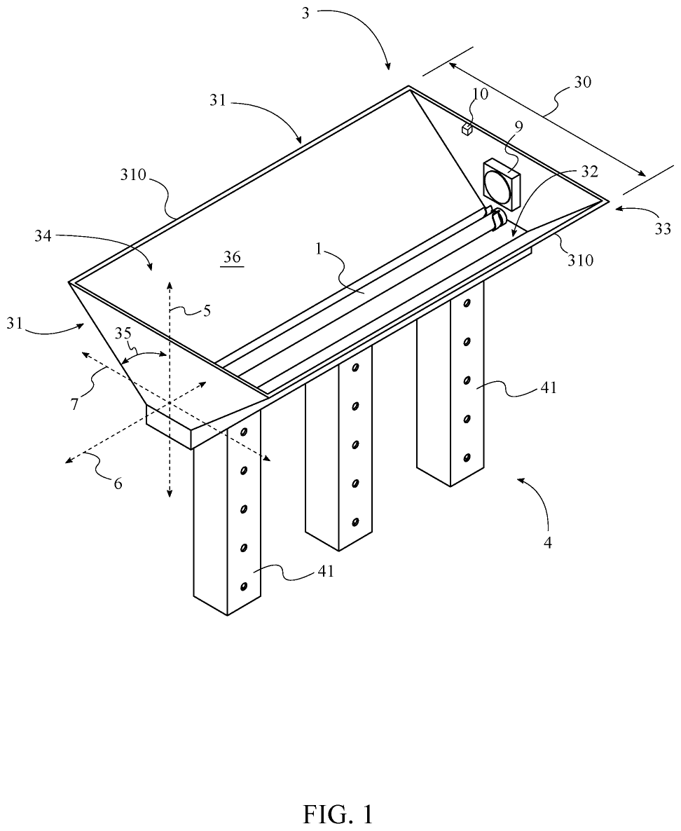

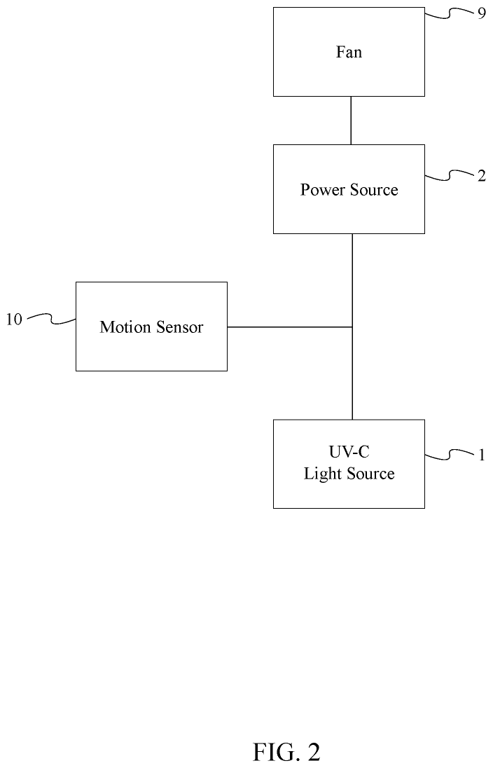

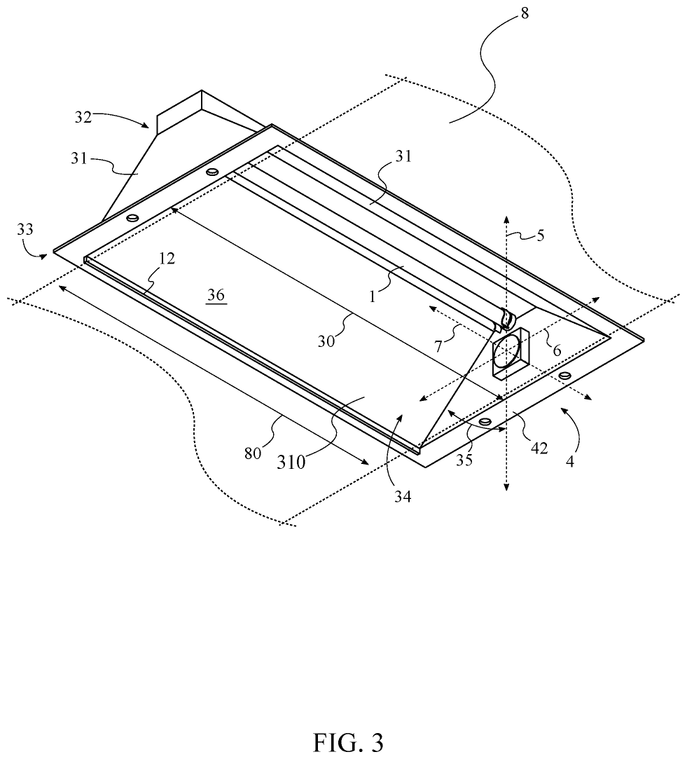

In general, referring to FIGS. 1-3, the present invention comprises a UV-C light source 1, a power source 2, a housing 3, and a mount 4. The housing 3 comprises a plurality of lateral walls 31, an apex end 32, a treatment end 33, and a treatment opening 34. The UV-C light source 1 is positioned within the housing 3 adjacent to the apex end 32, and is electrically connected to the power source 2. The treatment opening 34 is delineated by the plurality of lateral walls 31 at the treatment end 33. When installed onto a retail checkout counter, the treatment opening 34 is positioned adjacent to and spanning a width 80 of a conveyor belt 8 of the retail checkout counter as illustrated in FIG. 3, so that ultraviolet radiation emitted by the UV-C light source 1 falls upon the portion of the conveyor belt 8 positioned within the boundaries of the treatment opening 34 at any given time.

The plurality of lateral walls 31 extends between the apex end 32 and the treatment end 33. The housing 3 is closed at the apex end 32. A vertical direction 5 may be defined herein as oriented orthogonal to the treatment opening 34, wherein the apex end 32 and the treatment end 33 are positioned vertically opposite each other along the plurality of lateral walls 31.

In the preferred embodiment, the mount 4 is externally connected adjacent to the housing 3, wherein the mount 4 is configured to mount the housing 3 adjacent to a conveyor belt 8 of a retail checkout counter. A width 30 of the housing 3 should correspond to, or be slightly greater than, a width 80 of the conveyor belt 8, as shown in FIG. 3, wherein the width 30 of the housing 3 is oriented parallel to a lateral direction 7, and wherein the mount 4 is configured to position the treatment opening 34 adjacent to the conveyor belt 8. However, it should be noted that the mount 4 may take any desirable form in various embodiments, so long as the mount 4 facilitates the present invention being installed with the treatment opening 34 being positioned adjacent to the conveyor belt 8.

It is contemplated herein that in order to achieve optimal sanitation of a subject surface, object, or region through ultraviolent germicidal irradiation, various parameters may need to be specified and realized in the present invention. Such parameters may include, but are not limited to, intensity, propagation direction, wavelength, polarization, coherence, or other properties of light emitted by the UV-C light source 1, distance between the UV-C light source 1 and the conveyor belt 8 or other sanitization subject, and angle of incidence of light upon the subject. It is further contemplated that the engineering of such parameters in order to effectively sterilize a surface is a well known subject, and need not necessarily be specifically defined herein.

In some embodiments, the UV-C light source 1 is oriented parallel to the lateral direction 7. In some embodiments, the UV-C light source 1 is oriented perpendicular to the lateral direction 7, parallel to a longitudinal direction 6, which corresponds with the direction of travel of the conveyor belt 8. Both aforementioned orientations have advantages and drawbacks, and both may be realized in the present invention with equal conformity to the spirit and scope of the present invention. In some embodiments, the UV-C light is a light-emitting diode (LED) UV-C light source 1. In some embodiments, the UV-C light is a cold cathode UV-C light source 1. In some embodiments, the UV-C light source 1 is configured to emit UV-C light of a specified intensity. In some embodiments, the UV-C light source 1 is vertically separated from the conveyor belt 8 by a specified treatment distance. In some embodiments, for example, the specified treatment distance is three inches, though the specified treatment distance may vary in different embodiments according to the aforementioned engineering of parameters of the UV-C light source 1. The UV-C light source 1 may vary as desired in various embodiments so long as the UV-C light source 1 is configured adequately to inactivate microorganisms and sterilize the conveyor belt 8 or other subject surface, object, or region.

In the preferred embodiment of the present invention, the plurality of lateral walls 31 comprises a plurality of angled wall members 310, each of the plurality of angled wall members 310 being oriented at a desired UV reflection angle 35 to the vertical direction 5. Furthermore, each of the plurality of angled wall members 310 comprises an internal reflective surface 36. More particularly, in the preferred embodiment, each of the plurality of angled wall members 310 is constructed of a bright annealed steel material; more particularly, in the preferred embodiment, each of the plurality of angled wall members 310 is constructed of a bright annealed stainless steel material. In alternative embodiments, alternative means may be utilized to realize the internal reflective surface 36. For example, in some embodiments, the internal reflective surface 36 may be coated with any desirable reflective coating material, may be constructed of any desirably reflective material, or may be subject to any applicable treatment process which increases the reflectivity of the internal surface of the angled wall members 310. The desired UV reflection angle 35 is not specifically defined herein, but it may be understood that in the preferred embodiment, the desired UV reflection angle 35 is selected in order to maximize the amount 4 and/or intensity of UV-C light reflected by the plurality of angled wall members 310 from the UV-C light source 1 toward the treatment opening 34 in accordance with the aforementioned engineering of the UV-C light source 1.

Conveyor belts of retail checkout counters may vary in width, and one objective of the present invention is to be universally compatible with any such conveyor belts. To this end, in some embodiments, the housing 3 comprises a first housing portion 37 and a second housing portion 38, the first housing portion 37 and the second housing portion 38 being adjustably attached to each other along the lateral dimension. Thus, in order to install the present invention onto conveyor belt 8 of a retail checkout counter, a user may manually adjust the lateral position of the first housing portion 37 and the second housing portion 38 with respect to each other in order to fit over conveyor belts 8 of any width 80.

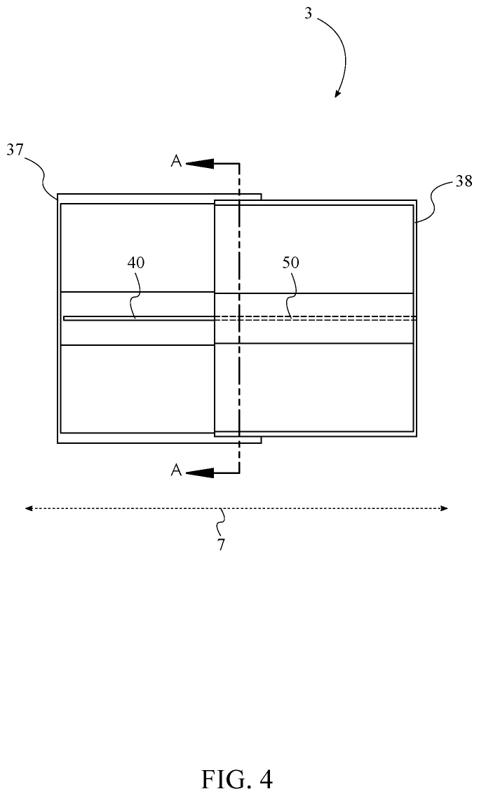

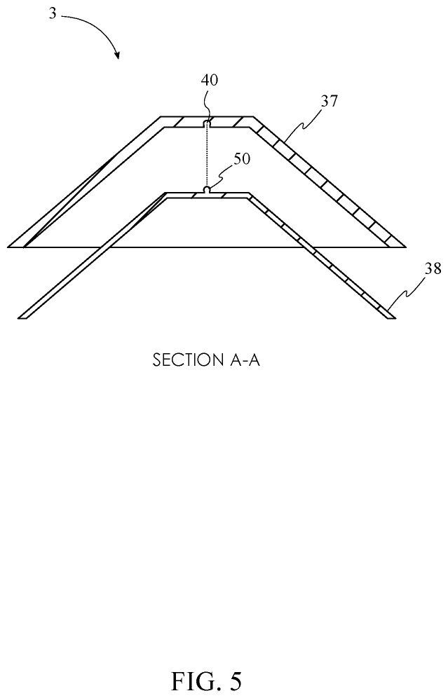

More particularly, in some embodiments as shown in FIG. 4-5, the first housing portion 37 comprises a first adjustment member 40, while the second housing portion 38 comprises a second adjustment member 50. The first adjustment member 40 and the second adjustment member 50 are each oriented parallel to the lateral direction 7, and the second adjustment member 50 is slidably positioned within the first adjustment member 40, such that the first housing portion 37 and the second housing portion 38 are slidably attached to each other along the lateral direction 7 through the first adjustment member 40 and the second adjustment member 50.

Some embodiments of the present invention may further comprise a fan 9 in order to mitigate any accumulation of heat within the housing 3 produced by the UV-C light source 1. In various embodiments, the fan 9 may be positioned in any desired location that allows the fan 9 to dissipate heat from within the housing 3. The fan 9 is electrically connected to the power source 2. In some embodiments, the fan 9 is internally connected to the housing 3. In some embodiments, the housing 3 may further comprise a fan 9 aperture traversing through one of the plurality of lateral walls 31, into which the fan 9 is installed in order to pull fresh air into the internal space of the housing 3. Some embodiments may further comprise one or more exhaust apertures traversing through one or more of the plurality of lateral walls 31 to allow continuous air flow through the housing 3.

It is one desired feature of the present invention that when the conveyor belt 8 ceases to move, the UV-C light source 1 continues to emit UV-C light for a specified period of time; for example, 20 minutes, or any other desirable period of time, after which the UV-C light source 1 is deactivated. Furthermore, it is desirable that the UV-C light source 1 automatically activate is motion of the conveyor belt 8 is detected. It is contemplated that various means may be realized in the present invention in order to accomplish this purpose; however, in some embodiments, the present invention may further comprise a motion sensor 10. The motion sensor 10 may be electrically connected between the power source 2 and the UV-C light source 1. Further, the motion sensor 10 may be positioned and configured to detect motion adjacent to the treatment opening 34, wherein the motion sensor 10 is configured to detect motion of a conveyor belt 8 of a retail checkout counter in the preferred embodiment. The motion sensor 10 may be further configured to deactivate the UV-C light source 1 is motion is not detected by the motion sensor 10 after a specified time period. Further, the motion sensor 10 may be configured to activate the UV-C light source 1 if motion is detected after the specified time period and/or after the UV-C light source 1 has been deactivated.

As previously mentioned, the mount 4 may be configured in any desirable manner to enable mounting the present invention to a retail checkout counter over its conveyor belt 8. In some embodiments, the present invention may be configured to position the treatment opening 34 adjacent to an underside of the conveyor belt 8, within a conveyor cabinet of the retail checkout counter. In such embodiments, as shown in FIG. 1, the mount 4 may comprise a plurality of legs 41, the plurality of legs 41 being perpendicularly and externally connected to the housing 3, opposite the UV-C light source 1 through the housing 3. It is further contemplated that other components and arrangements may be comprised in order for the mount 4 to facilitate installation of the present invention within the conveyor cabinet, under the conveyor belt 8.

Alternatively, in some embodiments, as shown in FIG. 3, the mount 4 may be configured to position the treatment opening 34 adjacent to the conveyor belt 8, external to and atop the conveyor cabinet of the retail checkout counter. To this end, in some embodiments, the mount 4 may comprise at least one mounting flange 42, each of the at least one mounting flange 42 being externally connected to the housing 3 adjacent to the treatment end 33. The housing 3 may be mounted atop the retail checkout counter over its conveyor belt 8 by affixing one or more fasteners through one or more fastener holes of the at least one mounting flange 42 and into a corresponding mounting surface of the retail checkout counter, or the housing 3 may be mounted atop the retail checkout counter through other suitable means.

In some embodiments, in addition to the sanitization effect provided through the UV-C light, the present invention may be further configured to clear physical debris from the conveyor belt 8. To this end, in some embodiments, the present invention may comprise a wiping member 12, as shown in FIG. 3. The wiping member 12 may be positioned in any suitable location to arrest any articles of physical debris traveling along the conveyor belt 8 by physically blocking their motion along the conveyor belt 8. In some embodiments, the wiping member 12 is positioned opposite the apex end 32 along the one of the plurality of lateral walls 31, wherein the one of the plurality of lateral walls 31 is positioned between the apex end 32 and the wiping member 12.

Although the invention has been explained in relation to its preferred embodiment, it is to be understood that many other possible modifications and variations can be made without departing from the spirit and scope of the invention as hereinafter claimed.

What is claimed is:

1. An ultraviolet light sanitation device for retail checkout counter conveyor belts comprising:
an ultraviolet-C (UV-C) light source;
a power source;
a housing;
a mount;
the housing comprising a plurality of lateral walls, an apex end, a treatment end, and a treatment opening;
the UV-C light source being positioned within the housing adjacent to the apex end;
the UV-C light source being electrically connected to the power source;
the treatment opening being delineated by the plurality of lateral walls at the treatment end;
the plurality of lateral walls extending between the apex end and the treatment end, wherein the housing is closed at the apex end, wherein a vertical direction is oriented orthogonal to the treatment opening, and wherein the apex end and the treatment end are positioned vertically opposite each other along the plurality of lateral walls;
the plurality of lateral walls comprising a plurality of angled wall members;
each of the plurality of angled wall members being oriented at a desired UV reflection angle to the vertical direction; and
the mount being externally connected adjacent to the housing, wherein the mount is configured to mount the housing adjacent to a conveyor belt of a retail checkout counter, wherein a width of the housing corresponds to a width of the conveyor belt, wherein the width of the housing is oriented parallel to a lateral direction, and wherein the mount is configured to position the treatment opening adjacent to the conveyor belt.

2. The ultraviolet light sanitation device for retail checkout counter conveyor belts as claimed in claim 1, wherein the UV-C light source is oriented parallel to the lateral direction.

3. The ultraviolet light sanitation device for retail checkout counter conveyor belts as claimed in claim 1, wherein the UV-C light source is oriented perpendicular to the lateral direction, parallel to a longitudinal direction.

4. The ultraviolet light sanitation device for retail checkout counter conveyor belts as claimed in claim 1, wherein the UV-C light source is a light-emitting diode (LED) UV-C light source.

5. The ultraviolet light sanitation device for retail checkout counter conveyor belts as claimed in claim 1, wherein the UV-C light source is a cold cathode UV-C light source.

6. The ultraviolet light sanitation device for retail checkout counter conveyor belts as claimed in claim 1, wherein the UV-C light source is configured to emit UV-C light of a specified intensity.

7. The ultraviolet light sanitation device for retail checkout counter conveyor belts as claimed in claim 1, wherein the UV-C light source is vertically separated from the conveyor belt by a specified treatment distance.

8. The ultraviolet light sanitation device for retail checkout counter conveyor belts as claimed in claim 7, wherein the specified treatment distance is three inches.

9. The ultraviolet light sanitation device for retail checkout counter conveyor belts as claimed in claim 1, wherein the each of the plurality of angled wall members comprises an internal reflective surface.

10. The ultraviolet light sanitation device for retail checkout counter conveyor belts as claimed in claim 1, wherein the each of the plurality of angled wall members is constructed of a bright annealed steel material.

11. The ultraviolet light sanitation device for retail checkout counter conveyor belts as claimed in claim 1, wherein the desired UV reflection angle is selected in order to maximize the amount of UV-C light reflected by the plurality of angled wall members from the UV-C light source toward the treatment opening.

12. The ultraviolet light sanitation device for retail checkout counter conveyor belts as claimed in claim 1 comprising:
the housing comprising a first housing portion and a second housing portion; and
the first housing portion and the second housing portion being adjustably attached to each other along the lateral direction.

13. The ultraviolet light sanitation device for retail checkout counter conveyor belts as claimed in claim 12 comprising:
the first housing portion comprising a first adjustment member;
the second housing portion comprising a second adjustment member;
the first adjustment member and the second adjustment member being oriented parallel to the lateral direction; and
the second adjustment member being slidably positioned within the first adjustment member, wherein the first housing portion and the second housing portion are slidably attached to each other along the lateral direction through the first adjustment member and the second adjustment member.

14. The ultraviolet light sanitation device for retail checkout counter conveyor belts as claimed in claim 1 comprising:
a fan;
the fan being internally connected to the housing; and
the fan being electrically connected to the power source.

15. The ultraviolet light sanitation device for retail checkout counter conveyor belts as claimed in claim 1 comprising:
a motion sensor;
the motion sensor being electrically connected between the power source and the UV-C light source; and
the motion sensor being configured to detect motion adjacent to the treatment opening, wherein the motion sensor is configured to detect motion of the conveyor belt of the retail checkout counter, wherein the motion sensor is configured to deactivate the UV-C light source if motion is not detected by the motion sensor after a specified time period, and wherein the motion sensor is configured to activate the UV-C light source if motion is detected after the specified time period.

16. The ultraviolet light sanitation device for retail checkout counter conveyor belts as claimed in claim 1 comprising:
the mount comprising a plurality of legs; and
the plurality of legs being perpendicularly and externally connected to the housing, opposite the UV-C light source through the housing, wherein the plurality of legs is configured to position the treatment opening adjacent to an underside of the conveyor belt, within a conveyor cabinet of the retail checkout counter.

17. The ultraviolet light sanitation device for retail checkout counter conveyor belts as claimed in claim 1 comprising:
the mount comprising at least one mounting flange;
each the at least one mounting flange being externally connected to the housing adjacent to the treatment end, wherein the at least one mounting flange is configured to position the treatment opening adjacent to the conveyor belt, external to and atop a conveyor cabinet of the retail checkout counter.

18. The ultraviolet light sanitation device for retail checkout counter conveyor belts as claimed in claim 1 comprising:
a wiping member; and
the wiping member being connected to one of the plurality of lateral walls adjacent to the treatment end.

19. The ultraviolet light sanitation device for retail checkout counter conveyor belts as claimed in claim 18 comprising:
the wiping member being positioned opposite the apex end along the one of the plurality of lateral walls, wherein the one of the plurality of lateral walls is positioned between the apex end and the wiping member.
